(19) United States
(12) Patent Application Publication
(10) Pub. No.: US 2010/0159505 A1
(43) Pub. Date: Jun. 24, 2010

(54) REAL-TIME MONITORING OF AGE PIGMENTS AND FACTORS RELATING TO TRANSMISSIBLE SPONGIFORM ENCEPHALOPATHIES AND APPARATUS

(75) Inventors: Thomas A. Casey, Ames, IA (US); Mark A. Rasmussen, McCallsburg, IA (US); Albin H. Gapsch, Melbourne, FL (US); Ricky Lee Flick, Lancaster, KY (US); Jacob W. Petrich, Ames, IA (US)

Correspondence Address: ERICKSON, KERNELL, DERUSSEAU & KLEYPAS, LLC, 800 W. 47TH STREET, SUITE 401, KANSAS CITY, MO, 64112, US

(73) Assignees: MEAT INSPECTION SYSTEMS, L.L.C., Bonita Springs, FL (US); IOWA STATE UNIVERSITY RESEARCH FOUNDATION, INC., Ames, IA (US); The United States, as represented by the Secretary of Agriculture, Washington, DC (US)

(21) Appl. No.: 12/715,028

(22) Filed: Mar. 1, 2010

Related U.S. Application Data
(62) Division of application No. 10/638,695, filed on Aug. 8, 2003.
(60) Provisional application No. 60/402,144, filed on Aug. 9, 2002.
(60) Provisional application No. 60/412,970, filed on Sep. 23, 2002.

(51) Int. Cl. C12Q 1/04; G01N 33/48

(52) U.S. Cl. 435/34; 435/40.5

(57) ABSTRACT

A fluorescent spectroscopic method and apparatus for real time direct detection of transmissible spongiform encephalopathies in central nervous system tissue by monitoring the fluorescence of intrinsic markers in the tissue by illuminating the tissue with UV or visible light having an appropriate wavelength, and the resulting emission spectra is detected and examined in the region from 350 to 650 nm. A higher intensity in this region is indicative of infected tissue. The apparatus and method would not interfere with existing slaughterhouse line speeds or procedures, and could be used on live animals.

### CROSS-REFERENCE TO RELATED APPLICATIONS

The present application is a division of U.S. patent application Ser. No. 10/638,695 Filed Aug. 8, 2003 which claimed the benefit of two previously filed co-pending Provisional Patent Applications, Ser. Nos. 60/402,144, filed Aug. 9, 2002; and 60/412,970, filed Sep. 23, 2002.

### GOVERNMENT INTERESTS

Funding for the work described herein was provided at least in part by the U.S. Federal Government, which may have certain rights in the invention.

### BACKGROUND OF THE INVENTION

1. Field of the Invention

The disclosed Invention relates to the field of fluorescent spectroscopy and more specifically, the present invention employs light to detect the presence of age pigment and factors related to transmissible spongiform encephalopathies present in the tissue of animals. More specifically, it involves the use of fluorescent spectroscopy to detect the presence of intrinsic markers in the central nervous system tissue.

2. Description of the Related Art

Recently, the rapid spread of bovine spongiform encephalopathy and its correlation with elevated occurrence of spongiform encephalophathies in humans has lead to a significant increase of interest in the detection of transmissible spongiform encephalopathies in non-human mammals. The tragic consequences of accidental transmission of these diseases (see, e.g., Gajdusek, Infectious Amyloids, and Prusiner Prions In Fields Virology. Fields, et al., eds. Lippincott-Ravin, Pub. Philadelphia (1996); Brown et al. (1992) Lancet, 340: 24-27), and the decontamination difficulties (Asher et al. (1986) pages 59-71 In: Laboratory Safety: Principles and Practices, Miller ed. Am. Soc. Microb.), and recent concern about bovine spongiform encephalopathy (British Med. J. 1995; 311: 1415-1421) underlie the urgency of having a diagnostic test that would identify humans and animals with transmissible spongiform encephalopathies.

The inability of the current art to adequately address this problem is shown by the continued failures to eliminate such materials from meat meant for consumption. For instance, Volume 4, Issue 30 of MEAT NEWS, dated Jul. 23, 2002 recounts a report by a French food agency that more than ten percent of the abbattoirs in France are failing to remove spinal cord from beef. Currently in the US, there is no regulation to prevent this material from entering the human food supply, as is required in Europe. In the US, however, there is a need for a method to identify specified risk material which could be used to prevent this material from entering the animal food supply through the rendering industry.

The transmissible spongiform encephalopathies (TSE) constitute a group of neuro-degenerative diseases. In humans these diseases include Creutzfeldt-Jakob disease (CJD), Gerstmann-Straussler-Scheinker syndrome, Fatal Familial Insomnia, and Kuru (see, e.g., Harrison's Principles of Internal Medicine, Isselbacher et al., eds., McGraw-Hill, Inc. New York, (1994); Medori et al. 1992 N. Engl. J. Med., 326: 444-9.). In animals the TSE's include sheep scrapie, bovine spongiform encephalopathy, transmissible mink encephalopathy, and chronic wasting disease of captive mule deer and elk (Gajdusek, (1990) Subacute Spongiform Encephalopathies: Transmissible Cerebral Amyloidoses Caused by Unconventional Viruses. Pp. 2289-2324 In: Virology, Fields, ed. New York: Raven Press, Ltd.).

TSEs are thought to be caused by the accumulation of abnormal, protease-resistant proteins called prions. These prions resist normal cell autophagy. Prions are localized in central nervous tissue like brain, eye, and spinal cord. It is for this reason that corneal transplants are a risk factor in transmission of CJD in humans, and biopsies of the nictating membrane of the eye are used to test for TSE infection in animals. Other protease-resistant compounds, collectively called lipofuscins (i.e., age pigments), also accumulate in central nervous tissue. It is also known that lipofuscins accumulate in the eye especially in the diseased eye (e.g., in the retinal pigment epithelium). An increase in lipofuscin accumulation is known to occur in human CJD victims and in other cases of experimental TSEs.

To date, the prior art has approached the detection of these diseases in human and non-human mammals through the use of assays. For example, U.S. Pat. No. 5,998,149 by Hsich et al, discloses improved assay methods involving detecting the presence or absence of 14-3-3 proteins in cerebrospinal fluid from the tested organism. Elevated levels of 14-3-3 are believe to be indicative of transmissible spongiform encephalopathies, in particular Creutzfeldt-Jacob disease in humans or mad cow disease in bovines).

Another approach involves immobilizing biological molecules, in particular nucleic acid strands, as set forth in U.S. Pat. No. 6,306,584 issued to Bamdad. Biological molecules immobilized at surfaces can be used in electron-transfer detection techniques in which a binding partner of a biological molecule is brought into proximity of the surface-immobilized biological molecule, an electrical potential created between the two biologically-binding species, and electron transfer through the species determined.

Another technique involves immobilizing a biological molecule such as a protein, DNA, etc. at a surface via a self-assembled monolayer, affecting the biological molecule via, for example, biological binding, inducing a change in conformation via a prion, etc., and detecting an electronic property change in the molecule via a change in impedance associated with an electronic circuit addressed by the biological molecule. These technique facilitates combinatorial array detection articles.

Another recent innovation for the detection of TSE involves detecting the presence or absence of 14-3-3 proteins in cerebrospinal fluid from the tested organism through the use of labels, including fluorescent labels. Such a method is set forth in U.S. Pat. No. 5,998,149, Method of detecting transmissible spongiform encephalopathies, by Hsich et al. Elevated levels of 14-3-3 are indicative of transmissible spongiform encephalopathies, in particular Creutzfeldt-Jacob disease in humans or mad cow disease in bovines. The method disclosed in the '149 patent involves conjugating the molecules of interest to signal generating compounds such as a fluorophore. Fluorescent compounds include fluorescein and its derivatives, rhodamine and its derivatives, dansyl, umbelliferone, etc. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. However, we note that the use of use of 14-3-3 proteins in cerebrospinal fluid as an indicator of TSE has been called into question in recent research.

Although the foregoing methods serve their purposes of detection, there is a need for a detection method and apparatus that is capable of safely, quickly and accurately detecting the presence of factors responsible for transmissible spongiform encephalopathies with fewer steps and less effort than available in the prior art.

A method which has been used in the past to detect contaminants on the surface of foods involves the use of fluorescent spectroscopy. For example, Alfano (U.S. Pat. No. 5,474,910) disclosed a method and apparatus for detecting biological molecules and microorganisms by irradiating the sample material with UV light at a wavelength between about 250 to 325 nm and measuring the resultant fluorescence. Alfano further disclosed that the process could be used for detecting the bacterial spoilage of food products, including meat and poultry. More recently, Waldroup and Kirby (U.S. Pat. No. 5,621,215) disclosed a method and apparatus for detecting the contamination of meat or poultry with ingesta or fecal material. As described therein, the meat or poultry is illuminated with UV light having a wavelength between about 320 to 420 nm, and examined for fluorescence, specifically UV fluorescence.

Similarly, in U.S. Pat. No. 5,914,247, issued to Casey et al, it is disclosed that a carcass is illuminated with UV or visible light having a wavelength effective to elicit fluorescence of feces at a wavelength between about 660 to 680 nm. The emission of fluorescent light having wavelengths between about 660 to 680 nm is an indication of the presence of ingesta or fecal material on the carcass.

We have now surprisingly found that certain techniques of fluorescent spectroscopy may also be employed to detect age pigments and factors responsible for transmissible spongiform encephalopathies. Such a technique can be used to not only identify the presence of such materials, but also for use in identifying the sources or means of contamination, and thus talking corrective steps to prevent contamination by similar means in the future, such as in the meat handling and processing industries.

### SUMMARY OF THE INVENTION

A method and apparatus are disclosed for the real-time direct detection of central nervous system tissue by monitoring the fluorescence of intrinsic markers contained in it. The method and apparatus of the present invention enables the indirect detection of age pigments and factors responsible for transmissible spongiform encephalopathies.

The present invention employs light to detect the presence of age pigment and factors related to transmissible spongiform encephalopathies present in the tissue of animals. More specifically, it involves the use of fluorescent spectroscopy to detect the presence of intrinsic markers in the central nervous system tissue.

In its simplest form, an object of interest is illuminated with UV or visible light having an appropriate wavelength, and the resulting emission spectra is detected for analysis. The resulting emission spectra is analyzed, in particular the intensity of the emission light is examined in the region from 350-650 nm. A higher intensity in this region is indicative of infected tissue.

In one application, the tissue of an animal is illuminated with UV or visible light having a wavelength anywhere in the range from approximately 300 to 550 nm, and then fluorescent light emissions in the range from approximately 400-900 nm are detected. The emission of fluorescent light having wavelengths anywhere in the range from 400-900 nm is an indication of the presence of age pigments or factors relating to TSEs on the carcass.

In its simplest form, the apparatus for practicing the invention includes an excitation source such as a lamp or laser for illuminating the surface with UV or visible light having a wavelength anywhere in the range from 300 to 550 nm and a detector for collecting the resulting fluorescent light emissions having a wavelength in the range of approximately 400-900 nm.

In accordance with this discovery, it is an object of this invention to provide an improved method and apparatus for detecting the presence of factors related to TSEs in meat or animal carcasses to improve the safety of the food supply.

Another object of the invention is to provide an improved high-speed method and apparatus which is capable of near real time detection of factors related to TSEs in meat or animal carcasses or even hamburger or similar homogenous materials, which would not interfere with existing slaughterhouse line speeds or procedures. Such a method and apparatus can also be used to identify the presence of such factors, thus allowing for corrective actions to be taken to prevent future recurrences.

Yet another object of the invention is to provide an improved method and apparatus for the detection of factors related to TSEs in meat or animal carcasses with high sensitivity and accuracy and which is substantially free from non-specific background interference.

Another object of the invention is to provide for a nonlethal and noninvasive means of determining if an animal is infected with TSEs. A rapid means of identifying TSE infections in living animals would be very useful for purposes of disease identification and eradication.

These and other objects and advantages of the present invention will become apparent from the following detailed description when viewed in conjunction with the accompanying drawings, which set forth the preferred embodiments of various aspects of the invention.

### BRIEF DESCRIPTION OF THE DRAWINGS

The preferred embodiments of the present invention will now be described, with respect to the drawings in which:

FIG. 1(A) depicts the excitation spectra of spinal cord tissue extract in a chloroform/methanol mixture with emission monochromator at three different wavelengths.

FIG. 1(B) depicts the emissions spectra of spinal cord tissue extracts in a chloroform/methanol mixture with three different excitation wavelengths.

FIG. 2(A) depicts a visible, non-fluorescence-based image of a meat substrate upon which are placed samples of spinal cord and feces.

FIG. 2(B) depicts the spinal cord shown in FIG. 2(A) imaged by means of lipofuscin fluorescence observed at 610 nm.

FIG. 2(C) depicts the feces shown in FIG. 2(A) imaged by fluorescence observed at 670 nm.

FIG. 3(A) depicts the fluorescence emission spectra of sheep brain tissue, both healthy and infected with scrapie, where the brain tissue has not been treated with formalin.

FIG. 3(B) depicts the emission spectra of sheep brain tissue, both healthy and infected with scrapie, where the tissue has been treated with formalin and fixed on a glass slide.

FIG. 4 is a schematic diagram of the disclosed process and apparatus.

FIG. 5 shows an alternative embodiment of the apparatus.

### DETAILED DESCRIPTION OF THE PREFERRED EMBODIMENTS

The present invention will now be described more fully hereinafter with reference to the accompanying figures. It will be understood that the components and steps of the presently preferred embodiment of the present invention, as generally described and illustrated herein, could be arranged and designed in a wide variety of different configurations. Thus, this invention may be embodied in many different forms and should not be construed as limited to the embodiments set forth herein. Rather, these embodiments are provided so that this disclosure will be thorough and complete, and will fully convey the scope of the invention to those skilled in the art, and will be merely representative of the presently preferred embodiments of the invention.

### EXAMPLES 1(A) AND 1(B)

The present invention can be understood with reference to the spectra of emission and excitation light that are detected when a sample of spinal cord tissue is tested under conditions of varying excitation light or varying emission light. In Examples 1(A) and 1(B), spinal cord tissue is placed in a mixture of chloroform to methanol at a 2:1 volumetric ratio for testing.

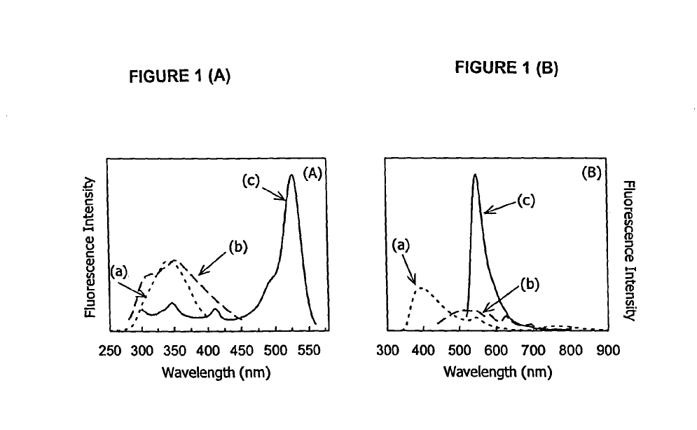

In Example 1(A), the sample of spinal cord tissue extract is exposed to a broad range of excitation wavelengths, and the result emissions are viewed through a monochromator set at a series of fixed emission wavelengths, including (a) 420 nm, (b) 480 nm, and (c) 580 nm. FIG. 1(A) depicts the resulting excitation spectra. For the sake of presenting the graphical data in a viewable form, the excitation spectra corresponding to the 420 nm emission has been scaled by a factor of twenty.

In Example 1(B), the same sample was exposed to a series of three discrete excitation wavelengths, including: (a) 330 nm (b) 420 nm, and (c) 500 nm. FIG. 1(B) depicts the resulting emission spectra.

As can be seen from the resulting graphs, variation in the excitation spectra with emission wavelength and the variation in emission spectra with excitation wavelength indicate that the extract in Examples 1(A) and 1(B) is very heterogeneous.

Application of the present invention for analytical methodology is further aided by the characteristic that there are materials in the extract that absorb strongly for wavelengths as long as 532 nm, which makes the present technology able to be used with commercially available Nd:YAG lasers. Furthermore, we have found that the farther into the red the excitation is tuned, the fewer intrinsic biological fluorophores are excited. Note that Example 1(B) results demonstrate detectable emissions even in the region from 700-900 nm. This characteristic lends the present invention useful for practical detection techniques as disclosed herein.

### EXAMPLE 2

The present invention can also be appreciated from the visual results of the application of one embodiment when it is used to excite a meat substrate (such as a piece of steak) upon which are placed samples of both spinal cord and feces. The steak and feces serve as controls in Example 2.

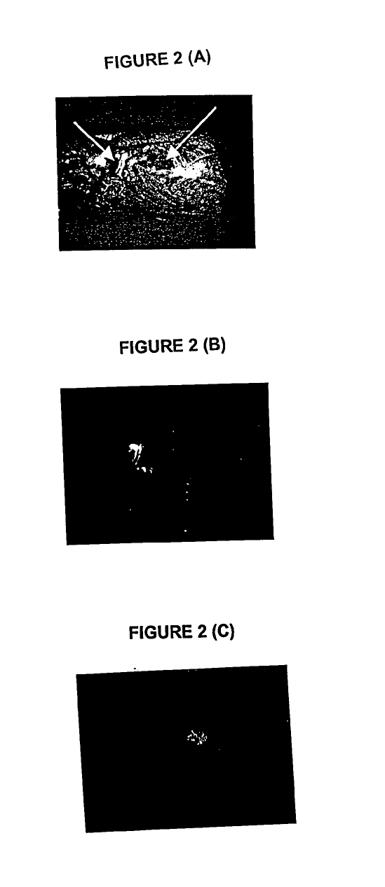

In FIG. 2(A), there is shown the visible, non-fluorescence-based image of the meat substrate. The spatial arrangement of the spinal cord and feces on the meat substrate are shown. The arrow at the left points to the spinal cord tissue; the one to the right, the feces.

Using a CHEM IMAGER 4000, Alphalnnotech Corp., San Leandro, Calif., in conjunction with an actinic blue aquarium light from Energy Savers Unlimited Inc., Harbor City, Calif. fitted with a 430-nm, 10-nm bandpass, interference filter from CVI Laser Corp., Albuquerque, N.M., the sample is exposed and the visual results are presented in FIGS. 2(B)-(C).

FIG. 2(B) depicts the meat sample when imaged by means of lipofuscin fluorescence observed at 610 nm (10-nm bandpass). As can be seen, the spinal cord sample fluoresces.

FIG. 2(C) depicts the same meat sample as seen when imaged by fluorescence observed at 670 nm (10-nm bandpass). The feces is seen to fluoresce.

FIGS. 2(A)-2(C) demonstrate an additional benefit of the present invention, namely that lipofuscin pigments in the CNS tissue of spinal cord permit it to be imaged at different wavelengths, and thus independently of feces.

The characteristic of the present process and apparatus which allows the spinal cord to be imaged without spectral interference from either the meat substrate or fecal contamination is significant for use of these pigments as analytical tools. For example, it is known that there are microbiological agents, products and other components already in feces with detectable and distinct optical characteristics. An example is the methanogenic bacteria, which are present in bovine feces and which will fluoresce when excited at certain wavelengths. There may also be a variety of plant pigments in addition to chlorophyll breakdown products, such as flavonoids, terpenoids, isoprenoids and carotenoids that may also contribute to fecal fluorescence. These obviously do not interfere with the lipofuscin signal shown in FIG. 2(B).

It is to be understood that the data in FIGS. 2(A)-(C) were obtained merely to provide a disclosure in the form of a visual demonstration of the concept of exploiting lipofuscin fluorescence, and were not obtained with optimized excitation or detection conditions. For example, 15-minute integration times were used to collect this data; whereas we have demonstrated both in the laboratory and in the field that such detection may be effected in real time.

### EXAMPLE 3

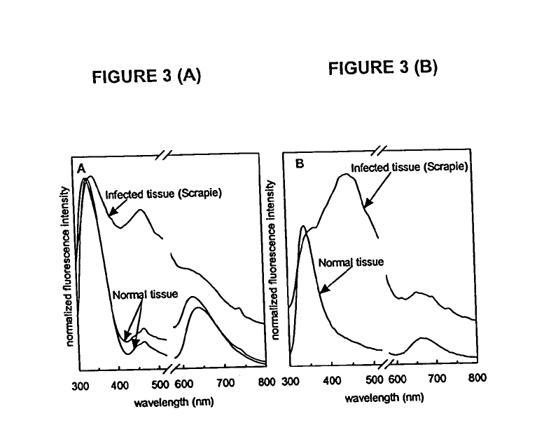

The literature indicates that abnormal TSE prions also display characteristic optical spectra. Our own preliminary data (FIG. 3) indicate that the fluorescent spectra of scrapic-infected sheep brain is substantially different from that of non-infected sheep brain. We hypothesize that this spectral difference is the result of altered lipofuscin and/or prion spectral properties.

In Example 3, sheep brain tissue, both healthy and infected with scrapie, a form of TSE to which sheep are susceptible, is excited at 280 nm using a front-faced excitation geometry. The resulting fluorescence emission spectra are shown in FIGS. 3(A)-(B), with all spectra normalized to unity at approximately 340 nm.

FIG. 3(A) depicts the spectra of untreated brain tissue not treated with formalin (a formaldehyde containing solution) and fixed on a glass slide, whereas FIG. 3(B) depicts the spectra of brain tissue treated with formalin and fixed on a glass slide. The region from 520 to 580 nm is omitted to eliminate the second order leakage of the excitation wavelength. The shape of the spectra was found to be same whether exciting at 280, 330 or 450 nm

FIG. 3 clearly indicates the red fluorescence, as discussed above in relation to Examples 1(A) and 1(B). An important feature of these spectra is that scrapie-infected tissue presents a spectrum that is fundamentally different than that of healthy tissue. The emission intensity of the infected sample is significantly higher in the region from 350-650 nm. This is the case no matter how one normalizes the data for the purposes of presentation.

### EXAMPLE 4

Lipofuscins and prions may serve as useful fluorescent markers, which are correlated with the occurrence of TSE's and can be detected by spectroscopy. The eye may serve as a useful diagnostic tissue worthy of examination because it is the one site where the central nervous system can be accessed by external, noninvasive means.

The objective of this technology is to employ fluorescence detection of marker pigments in the eye to develop a real time, noninvasive, technology for detecting TSE-related diseases in living animals for purposes of disease identification, eradication, and prevention. We have already observed (FIG. 3) that scrapie-infected tissue presents a spectrum that is fundamentally different than that of healthy tissue.

Based on the results in FIG. 3, we believe that the most promising wavelengths to be exploited for use in conjunction with TSE detection in the eye are in the regions of 580-620 nm and 700-900 nm. Although the emission in the 700-900 nm region seems to be weaker than that from the 580-620 nm region, the reduction in intensity may be compensated by the fact that no intrinsic biological chromophores exist so far in the red; and it is possible that the signal-to-noise of the detector may be greatly enhanced by exploiting this spectral region.

In a particular preferred embodiment, living animals such as cattle or sheep would be restrained and their eyes would be optically scanned for characteristic spectral patterns that are unique and indicative of TSEs. A modified scanning laser opthalmoscope or other suitable optical equipment which is tuned to the characteristic excitation and emission wavelengths could be used to scan the eyes of living animals for indication of TSE disease, such as the Eye Q of the Canon Retinal Imaging Products line, Canon USA Inc., One Canon Plaza, Lake Success, N.Y. Without being limited thereto, eye scanning may also be effected using the techniques described by Cabib (U.S. Pat. No. 6,419,361, the contents of which are incorporated by reference herein.

### Meat Processing Industry Application

The present invention has application in both the analytical laboratory and the high-speed meat processing context. At a typical slaughterhouse, testing of the carcasses is conducted at one or more stations, during transport along the line, or soon after completion of slaughter. As the carcasses pass the testing stations, they may be illuminated and any fluorescent light emitted therefrom detected as described herein.

The slaughter of beef cattle typically includes the following steps: the animal is rendered unconscious, shackled and hoisted onto a moving rail or line, exsanguinated, skinned (manually or in combination with mechanical hide pullers), and decapitated. The ends of the digestive tract may be tied off (to prevent contamination) prior to evisceration which is conducted using a deep, midline abdominal incision. The entrails are then removed onto a conveyor for inspection and further processing or disposal. The eviscerated carcass may then be split into halves by cutting longitudinally through the spinal column. These halves are inspected for quality and for nonedible defects (e.g. tumors). Those that pass inspection are then weighed and hung in a chiller room for approximately 24 hours before further processing or shipment. Testing may be conducted during or upon completion of any of the above-mentioned steps, or even after slaughter.

In a particularly preferred embodiment, ruminant (e.g. bovine) carcasses will be tested for contamination prior to chilling of the split carcasses, usually within about 1 hour after splitting or about 1 to 2 hours after initiation of slaughter. Other preferred sites for testing include prior to, during or after skinning or evisceration/gutting. Upon detection of factors relating to TSE, the carcass may be properly discarded, or cleaned of offending material. Such identification may also be used to detect the presence of factors relating to TSE in hamburger or other homogenous materials.

In addition to allowing the user to distinguish infected tissue from non-infected tissue, and thus locate such matter, the present invention also permits the operator of a slaughterhouse or any party in the meat processing cycle to take steps to prevent specified risk materials from getting into a meat product in the first place. For example, once a risk factor is determined to be present during processing, upstream processing procedures can be examined and corrective actions can be taken to prevent similar instances of future contamination.

In one embodiment, testing incorporates phase sensitive or phase lock (lockin) techniques. Testing in this manner allows quantitative measurements to be made in real time and in ambient light conditions, without using a light-tight testing chamber. The skilled practitioner will recognize that other spectroscopic techniques such as boxcar or gated integration techniques may also be used. In accordance with phase sensitive techniques, the excitation light is modulated at a predetermined frequency, for example, by mechanically chopping the light beam with a spinning wheel. Or, the excitation source may be light emitting diodes controlled by high frequency pulse generators. This modulated excitation source will in turn yield fluorescence from a contaminated carcass that is modulated at the same frequency. The fluorescence signals emitted from the carcass are then selectively detected at the same frequency as the modulated excitation light; only emitted fluorescent light at the same frequency is detected and amplified. Hence, no fluorescence signals due to stray or ambient light are detected. A detailed description of phase sensitive spectroscopic techniques which are suitable for use herein may be found, for example, in Horowitz and Hill (The Art of Electronics, Cambridge University Press, Cambridge, pp. 628-631), Shoemaker et al. (Experiments in Physical Chemistry, 5th ed., McGraw-Hill, New York, p. 737), Zanowsky (1996, Laser Focus World, 32:135-137), Ingle and Crouch (Spectrochemical Analysis, Prentice-Hall, New York, 1988, especially at pp. 125, 410, and 526), Fleming (Chemical Applications of Ultrafast Spectroscopy (Oxford Press, New York, 1986, especially at chapter 3), and Schreiber (1986, Photosynthesis Research, 9:261-272), the contents of each of which are incorporated by reference herein.

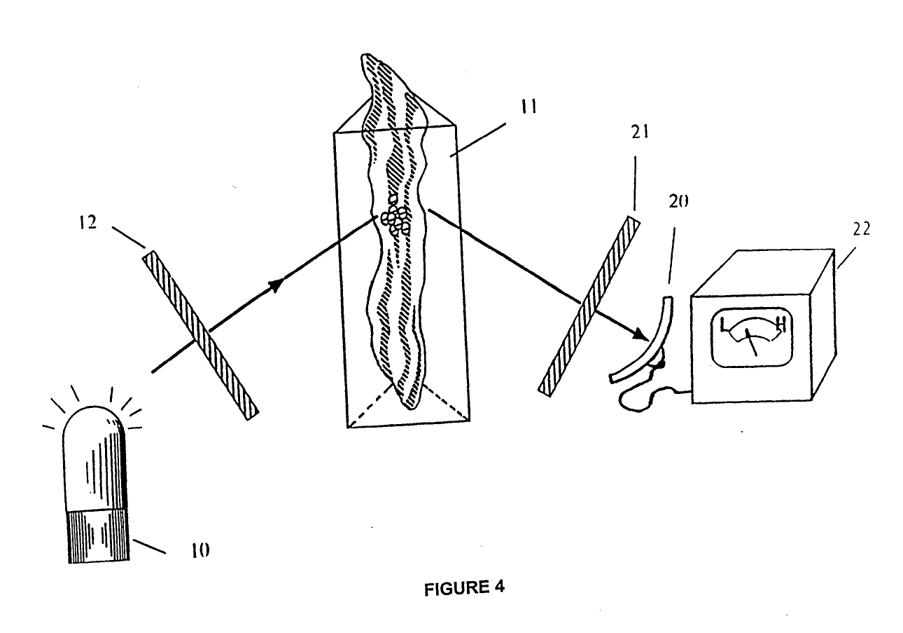

As shown in FIG. 4, the apparatus of the invention includes an excitation light source (10) which illuminates the surface of carcass (11) or other object to be examined. For use herein, the excitation light source of the present invention should emit electromagnetic radiation which is effective to elicit or cause the fluorescence of the factors relating to TSE at a wavelength between about 400 to 900 nm. A variety of coherent or incoherent excitation light sources are suitable for use herein, and include but are not limited to lasers, light emitting diodes (LED's), and arc lamps, and the light source may be continuous or pulsed. However, lasers emitting at an appropriate excitation wavelength are generally preferred. An optional optical filter (12) is preferably provided which permits only light of the selected excitation wavelength to pass through onto the carcass (11) or other object to be examined.

Fluorescent light emitted from the surface of the carcass (11) is detected using a photodetector (20) sensitive to at least 400-900 nm light. Without being limited thereto, suitable photodetectors (20) for use herein include photodiode detectors, photomultipliers, amplifiers or image intensifiers, CCD cameras, and photocathodes and microchannel plates (i.e. “Night vision” technology). One or more optical filters (21) are preferably positioned between the carcass and the photodetector (20) to selectively transmit light in the range of about 400-900 nm light while preventing transmission of back-scattered excitation light. Filters (21) are preferably effective to remove wavelengths of light less than about 400 and greater than about 900 nm.

In one embodiment, the photodetector (20) may include an image intensifier having a viewing lens or screen (22) mounted on the output thereof to allow for direct visual detection of fluorescence by the operator. In the alternative or in addition to the viewing eyepiece or screen, the output signal from the photodetector (20) may be relayed to a recording instrument, such as an oscilloscope or printer for presenting a graphical display of fluorescent spectra intensity. In yet another alternative, the photodetector (20) may be in communication with a signal generator (not shown) for generating and displaying a discard signal when the fluorescent intensity at the measured 400-900 nm range has exceeded a predetermined threshold value. Signals may include for example, audible alarms, visible lights or LEDs, or any combination of the above.

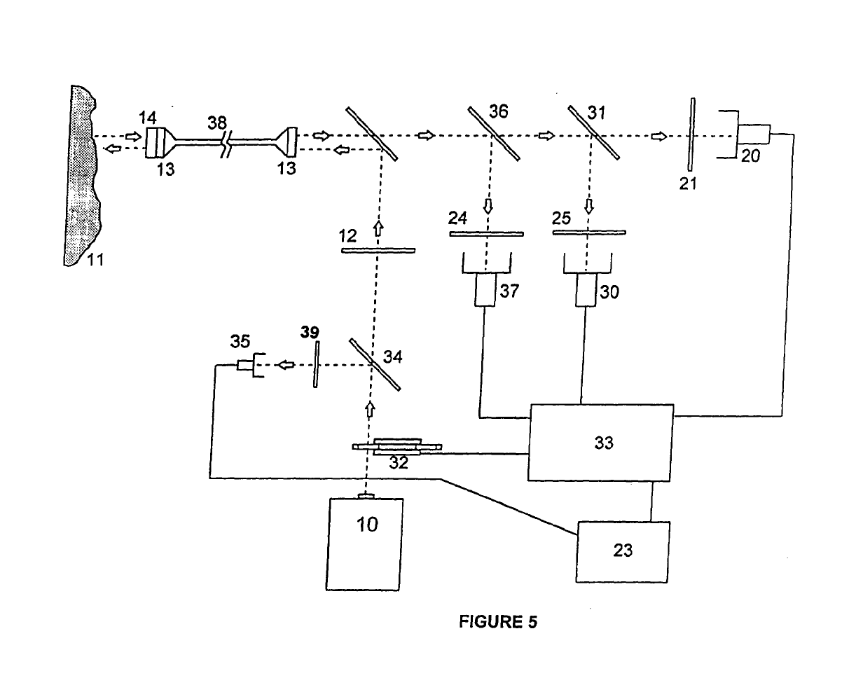

An optional microprocessor based control unit (23) (shown in FIG. 5) having conventional interface hardware may be provided for receiving and interpreting the signals from the photodetector, and manipulating data as described above. The signal generator (not shown), described above, may be provided in communication with the microprocessor (23) rather than photodetector (20). The microprocessor (23) may also be used for automated control of carcass testing, including automated scanning of carcasses, and/or directing carcasses found positive for factors related to TSE to be discarded.

A variety of optional modifications which may be made to the apparatus for use in the preferred embodiments are also within the scope of this invention and shown in FIG. 5. For instance, a photodetector (30) and cooperating dichroic mirror (31) may be provided for measurement intensity of baseline fluorescence for improving contrast as described hereinabove. Where quantitative measurements are to be made using phase sensitive technology, the apparatus may also include a frequency modulator such as chopper (32), lockin amplifier or gated integrator (33) in communication with chopper (32) and photodetectors (20 and 30), as well as beam splitter (34) and photodetector (35) for measuring the intensity of excitation light. Microprocessor (23) may then calculate the intensity of the emitted fluorescence at 400-900 nm from the phase shift and excitation light intensity data. Interference filters (25 and 21) can be placed before the photodetectors to reduce the stray light and increase the signal-to-noise ratio observed. Excitation light source (10) may also be equipped to provide an infrared beam which can be used as a reference for scattered light intensity from the carcass and as a distance probe from the carcass. In this event, an additional IR reflecting dichroic mirror (36) and IR photodetector (37) fitted with an IR interference filter (24) are included.

The apparatus may be constructed as a hand-held device for manual testing, or as a fixed pass-through box or station positioned along the slaughterhouse line for automated testing. Fiber optics (38), as shown in FIG. 5, may be provided for directing excitation light onto the carcass and/or collecting emitted fluorescent light. The fiber optic may be fitted with lenses (13) to collimate the laser beam to a given size on the carcass and to assist in the collection of emitted light. The fiber optic may also be fitted with a disposable tip (14) to gauge the distance between the excitation source and the carcass reproducibly and accurately. This is beneficial when quantifying contamination on the carcass.

What is claimed and desired to be secured by Letters Patent is as follows:

1. A method for detecting the presence of central nervous system tissue of an animal on a substrate comprising the steps of:
a) illuminating the substrate with light having a wavelength effective to elicit fluorescence from any central nervous system tissue of an animal thereon, said fluorescence at a wavelength between 400 to 900 nm; and
b) detecting fluorescent light emission from the central nervous system tissue of an animal at a wavelength between about 400 to 900 nm, wherein detection of fluorescent light emission at said wavelength between about 400 to 900 nm is an indication of the presence of central nervous system tissue of an animal on the substrate.

2. The method of claim 1, wherein said illuminating light is at a wavelength between about 300 to 550 nm.

3. The method of claim 1 wherein said substrate comprises meat.

4. The method of claim 1 wherein said substrate comprises an animal carcass.

5. The method of claim 4 wherein said illuminating and said detecting are conducted during or after slaughter of said animal.

6. The method of claim 5 wherein said illuminating and said detecting are conducted within about 2 hours after initiation of slaughter of said animal.

7. The method of claim 5 wherein said illuminating and said detecting are conducted within about 1 hour after the splitting of said carcass during slaughter.

8. The method of claim 1 wherein said detecting is repeated at different wavelengths between about 400 to 900 nm.

9. The method of claim 1 wherein said fluorescent light emission is detected at a wavelength of 420 nm.

10. The method of claim 1 wherein said fluorescent light emission is detected at a wavelength of 610 nm.

11. A method for detecting the presence of factors related to transmissible spongiform encephalopathies in the central nervous system tissue of an animal comprising the steps of:
a) illuminating the central nervous system tissue of an animal with light having a wavelength effective to elicit fluorescence from the factors related to transmissible spongiform encephalopathies said fluorescence at a wavelength between 400 to 900 nm; and
b) detecting fluorescent light emission from the central nervous system tissue of an animal at a wavelength between about 400 to 900 nm, wherein detection of fluorescent light emission at said wavelength between about 400 to 900 nm is an indication of the presence of factors related to transmissible spongiform encephalopathies in the central nervous system tissue of an animal.

12. The method of claim 11, wherein said illuminating light is at a wavelength between about 300 to 550 nm.

13. A method for detecting the presence of factors related to transmissible spongiform encephalopathies in an eye of a live animal using a system comprising the steps of:
a) illuminating the eye of a live animal with light having a wavelength effective to elicit fluorescence from the factors related to transmissible spongiform encephalopathies said fluorescence at a wavelength between 400 to 900 nm; and
b) detecting fluorescent light emission from the central nervous system tissue of the eye of a live animal at a wavelength between about 400 to 900 nm, wherein detection of fluorescent light emission at said wavelength between about 400 to 900 nm is an indication of the presence of factors related to transmissible spongiform encephalopathies in the central nervous system tissue of an animal.

14. The method of claim 13, wherein said illuminating light is at a wavelength between about 300 to 550 nm.
